(19) United States
(12) Patent Application Publication
(10) Pub. No.: US 2016/0237402 A1
(43) Pub. Date: Aug. 18, 2016

(54) Methods and Compositions for Ex Vivo Generation of Developmentally Competent Eggs from Germ Line Cells Using Autologous Cell Systems

(71) Applicant: NORTHEASTERN UNIVERSITY, Boston, MA (US)

(72) Inventors: Jonathan L. TILLY, Windham, NH (US); Dori C. WOODS, Londonderry, NH (US)

(21) Appl. No.: 15/026,163

(22) PCT Filed: Oct. 7, 2014
(86) PCT No.: PCT/US2014/059570

§ 371 Date: Mar. 30, 2016

Related U.S. Application Data
(60) Provisional application No. 61/887,569, filed on Oct. 7, 2013.

(51) Int. Cl. C12N 5/075; C12N 5/071; A61K 35/54

(52) U.S. Cl. CPC C12N 5/0609; A61K 35/54; C12N 5/0682; C12N 2500/90; C12N 2501/115; C12N 2501/11; C12N 2500/44; C12N 2501/13; C12N 2501/235; C12N 2506/02; C12N 2502/04; C12N 2510/00

(57) ABSTRACT

The present technology provides for methods for the directed differentiation of multi-potent cells, female germ line stem cells, or oogonial stem cells into oocytes, granulosa cells and/or granulosa precursor cells, i.e.,“synthetic granulosa cells.” The synthetic granulosa cells are useful in methods for growth and maturation of follicles or follicle-like structures and immature oocytes. Additionally, the synthetic granulosa cells are useful in methods of increasing ovarian derived hormones and growth factors in a subject in need thereof.

### CROSS-REFERENCE TO RELATED APPLICATION

This application claims priority to U.S. Provisional Application No. 61/887,569 filed Oct. 7, 2013, the content of which are incorporated herein by reference in its entirety.

### GOVERNMENT SUPPORT

The present technology was made with U.S. Government support under grant R37-AG012279 and F32-AG034809 awarded by the National Institutes of Health. The U.S. Government has certain rights in the present technology.

### BACKGROUND

Approximately 7,000,000 couples suffer from infertility in the USA, yet, only around 150,000 cycles of in vitro fertilization (IVF) are performed each year and these limited numbers reflect some couples going through the procedure twice in the same year. The large drop-off between those in need and those in pursuit of solutions to infertility (viz. less than 2% of infertile couples actually undergo assisted reproduction) is due to factors other than the generally high cost of infertility treatments. Notably, many women are not considered “good candidates” for IVF since they will fail to generate eggs in response to current hormonal injection protocols used to suppress and then hyperstimulate the ovaries for egg retrieval. Examples of women who are not considered good candidates for IVF include woman at advanced maternal ages who have a severely diminished population of immature egg cells (oocytes) remaining in their ovaries, or women who exhibit premature ovarian failure (POF)/premature ovarian insufficiency (POI) for a variety of reasons including, but not limited to, genetic causes, immunological (autoimmune) abnormalities, or prior exposure to cytotoxic therapies, which damage the ovaries (for example young girls and reproductive age women treated for cancer).

Ovarian failure, and the resulting menopause, occurs due to a loss of ovarian follicles, each of which are composed of a single oocyte surrounded by supportive somatic cells termed granulosa cells. In addition to serving as the primary endocrine producing structures in the ovaries, follicles are required to support development and maturation of the enclosed oocyte. Without granulosa cell support, newly formed oocytes will quickly die. With this loss of follicles and the steroid producing ovarian granulosa cells comes a loss of fertile potential and a diminished ability to produce steroid hormones, the latter of which results in a profound detrimental effect on women's health, impacting not only reproductive organs and tissues but bone, brain, and the cardiovascular system, among others. The net result is a decline in bone density and cognitive function with age, as well as an increase in cardiovascular diseases (CVDs), which are the leading causes of death in women worldwide.

### SUMMARY

In one aspect, the present technology provides methods for directed differentiation of multi-potent cells into granulosa cells and/or granulosa precursor cells, the method including: culturing multi-potent cells in culture conditions that direct the multi-potent cells to differentiate to granulosa cells and/or granulosa precursor cells, wherein the culture conditions comprise the absence of MEFs and LIF and the presence of a GSK inhibitor.

In some embodiments, the culture conditions further comprise the presence of bone morphogenetic protein (BMP4) and/or retinoic acid (RA).

In some embodiments, multi-potent cells contain a granulosa cell specific reporter, wherein expression of the granulosa cell specific reporter is indicative of a cell that is a granulosa cell or a granulosa cell precursor.

In some embodiments, the GSK-3 inhibitor is selected from the group consisting of SB216763, BIO, CHIR99021, lithium chloride (LiCl), maleimide derivatives, staurosporine, indole derivatives, paullone derivatives, pyrimidine and furopyrimidine derivatives, oxadiazole derivatives, thiazole derivatives, heterocyclic derivatives, and a combination thereof.

In some embodiments, the method also includes contacting the multi-potent cells with growth factors or activators of signaling pathways for granulosa cell specification.

In some embodiments, the growth factors or activators of signaling pathways for granulosa cell specification are one or more of bFGF, Jaggedl, or Jagged2.

In another aspect, the present technology provides methods for directed differentiation of multi-potent cells into granulosa cells and/or granulosa precursor cells, the method including: culturing multi-potent cells in culture conditions that direct the multi-potent cells to granulosa cells and/or granulosa precursor cells, wherein the conditions comprise the absence of MEFs and LIF and the presence of a GSK inhibitor, wherein the multi-potent cells are engineered to contain one or more inducible granulosa cell-specific genes; inducing expression of the one or more ovarian granulosa cell-specific genes; and forming synthetic granulosa cells.

In some embodiments, the method also includes culturing the multi-potent cells in the presence of bone morphogenetic protein (BMP4) and/or retinoic acid (RA).

In some embodiments, the multi-potent cells contain a granulosa cell specific reporter, wherein expression of the granulosa cell specific reporter is indicative of a cell that is a granulosa cell or a granulosa cell precursor.

In some embodiments, the one or more inducible granulosa cell-specific genes is selected from the group consisting of forkhead box L2 (Fox12), wingless type MMTV integration site family, member 4 (WNT4), Nr5a1, Dax-1, ATP-binding cassette, subfamily 9 (Abca9), acetyl-Coenzyme A acyltransferase 2 (mitochondrial 3-oxoacyl-Coenzyme A thiolase; Acaa2), actin, alpha 2, smooth muscle, aorta (Acta2), a disintegrin-like and metallopeptidase (reprolysin-like) with thrombosin type 1 motif, 17 (Adamts17), ADAMTS-like 2 (Adamts12), AF4/FMR2 family, member 1 (Aff1), expressed sequence AI314831 (AI314831), Aldo-keto reductase family 1, member C14 (Akr1c14), aldo-keto reductase family 1, Notch2, and member C-like (Akr1c1).

In another aspect, the present technology provides an ex vivo artificial ovarian environment, the artificial ovarian environment including: synthetic granulosa cells, wherein the synthetic granulosa cells are generated using anyone of the above methods; oocyte precursor cells; and ovarian tissue. In some embodiments, the synthetic granulosa cells, the oocyte precursor cells, and ovarian tissue are autologous.

In another aspect, the present technology provides methods for making a mature follicle and a mature oocyte, the method including: directing differentiation of multi-potent cell to granulosa cells and/or granulosa precursor cells (synthetic granulosa cells) using any one of the above method for making granulosa cells and/or granulosa precursor cells; combining the synthetic granulosa cells with oocyte precursor cells, and ovarian tissue; and culturing the combination of synthetic granulosa cells with oocyte precursor cells, and ovarian tissue in conditions suitable to form the mature follicle and mature oocyte.

In some embodiments, the conditions suitable to form the mature follicle and the mature oocyte include the presence of follicle stimulating hormones (FSH) and/or luteinizing hormone (LH).

In another aspect, the present technology provides growth and maturation of follicles and immature oocytes in ovarian tissue in a subject in need thereof, comprising contacting ovarian tissue with granulosa cells and/or granulosa precursor cells (synthetic granulosa cells), wherein the synthetic granulosa cells are generated using anyone of the above methods.

In some embodiments, the synthetic granulosa cells contact the ovarian tissue in vivo.

In some embodiments, the synthetic granulosa cells are directly injected into the subject's ovarian tissue.

In some embodiments, the subject in need thereof suffers from one of more of the following issues selected from the group consisting of having trouble conceiving, undergoing infertility treatment, undergoing in vitro fertilization, has been treated for cancer, and has been subjected to cytotoxic therapies.

In another aspect, the present technology provides methods for increasing levels of one or more ovarian derived hormones or growth factors in a subject in need thereof, the method including: directing differentiation of multi-potent cell to granulosa cells and/or granulosa precursor cells (synthetic granulosa cells), wherein the synthetic granulosa cells are generated using anyone of the above methods; isolating an enriched population of synthetic granulosa cells based on expression of a granulosa cell specific reporter; and administering an effective amount of the enriched population of synthetic granulosa cells to the subject, wherein the granulosa cells or granulosa cell precursors secrete one or more ovarian derived hormones and growth factors, and wherein after administration of the synthetic granulosa cells the subject displays elevated levels of one or more ovarian derived hormones or growth factors as compared to the subject before administration of the enriched population of synthetic granulosa cells.

In some embodiments, the method also includes stimulating the synthetic granulosa cells to secrete ovarian derived hormones.

In some embodiments, the ovarian derived hormones are selected from the group consisting of: estradiol, estriol, estrone, pregnenolone, and progesterone.

In some embodiments, the granulosa cells or granulosa cell precursors are stimulated to secrete ovarian derived hormones by follicle-stimulating hormone (FSH), 8-Bromoadenosine 3′,5′-cyclic monophosphate (8-br-cAMP), and luteinizing hormone (LH).

In some embodiments, the population of synthetic granulosa cells are autologous to the subject. In some embodiments, the subject is human.

In another aspect, the present technology provides an ex vivo method for producing mature follicles and mature oocytes, the method including: combining synthetic granulosa cells, oocyte precursor cells, and ovarian tissue; and culturing the combination of synthetic granulosa cells, oocyte precursor cells, and ovarian tissue in conditions sufficient to produce mature follicles and a mature oocyte, wherein the synthetic granulosa cells are generated using anyone of the above methods and wherein the synthetic granulosa cells, the oocyte precursor cells, and the ovarian tissue are autologous.

In some embodiments, the oocyte precursor cells are derived from multi-potent cells, female germ line stem cells, or oogonial stem cells (OSCs). In some embodiments, the oocyte precursor cells are primordial germ cells, female germ line stem cells, or oogonial stem cells.

In some embodiments, the multi-potent cells, female germ line stem cells, or oogonial stem cells are genetically modified to correct for a gene defect. In some embodiments, the multi-potent cells, female germ line stem cells, or oogonial stem cells are genetically modified using one or more techniques selected from the group consisting of electroporation, direct injection of encoding mRNAs, lipid based transfection, retroviral transduction, adenoviral transduction, lentiviral transduction, CRISPR/Cas9, TALENs, zinc finger nucleases (ZFNs), engineered meganucleases, and site directed mutagenesis.

In some embodiments, the invention provides a method for developing genetically modified mature oocytes for a subject diagnosed with a genetic disease or disorder comprising: genetically modifying multi-potent cells or oocyte precursor cells (e.g., female germ line stem cells or oogonial stem cells) from the subject to correct a gene defect; culturing the genetically-modified multi-potent cells in conditions sufficient to produce oocyte precursor cells; combining the genetically modified oocyte precursor cells, without or with synthetic granulosa cells, and with ovarian tissue, wherein the synthetic granulosa cells, if utilized, are generated using anyone of the above methods and wherein the synthetic granulosa cells, if utilized, and ovarian tissue are autologous to the subject; and culturing the combination of oocyte precursor cells and ovarian tissue, without or with synthetic granulosa cells, in conditions sufficient to produce mature follicles and a mature oocyte, wherein the mature oocyte does not carry the genetic disease.

In some embodiments, the multi-potent cells, female germ line stem cells, or oogonial stem cells are genetically modified using one or more techniques selected from the group consisting of electroporation, direct injection of encoding mRNAs, lipid based transfection, retroviral transduction, adenoviral transduction, lentiviral transduction, CRISPR/Cas9, TALENs, zinc finger nucleases (ZFNs), engineered meganucleases, and site directed mutagenesis.

In another aspect, the present technology provides a method for producing mature oocytes ex vivo for using in in vitro fertilization, the method including combining synthetic granulosa cells, oocyte precursor cells, and ovarian tissue; and culturing the combination of synthetic granulosa cells, oocyte precursor cells, and ovarian tissue in conditions sufficient to produce mature follicles and a mature oocyte, wherein the synthetic granulosa cells are generated using anyone of the above methods and wherein the synthetic granulosa cells, the oocyte precursor cells, and the ovarian tissue are autologous.

In some embodiments, the oocyte precursor cells are derived from multi-potent cells, female germ line stem cells, or oogonial stem cells. In some embodiments, the oocyte precursor cells are primordial germ cells, female germ line stem cells, or oogonial stem cells. In some embodiments, the multi-potent cells, female germ line stem cells, or oogonial stem cells are genetically modified to correct for a gene defect. In some embodiments, the multi-potent cells, female germ line stem cells, or oogonial stem cells are genetically modified using one or more techniques selected from the group consisting of electroporation, direct injection of encoding mRNAs, lipid based transfection, retroviral transduction, adenoviral transduction, lentiviral transduction, CRISPR/Cas9, TALENs, zinc finger nucleases (ZFNs), engineered meganucleases, and site directed mutagenesis.

In some embodiments, the method also includes freezing the mature oocyte.

### BRIEF DESCRIPTION OF THE DRAWINGS

FIG. 1A is a graph that shows that oogonial stem cells (OSCs) persist in aged mouse ovaries. Germ line stem cells (also referred to as oogonial stem cells or OSCs) were isolated from C57B1/6 mice ovaries using anti-Ddx4 antibodies coupled with fluorescence-activated cell sorting (FACS) (Woods and Tilly, Nature Protocols, 8:966-88 (2013)), wherein the mice were in the age range of 3, 6, 10, 15, and 20 months.

FIG. 1B shows examples of immature oocytes generated in cultures of OSCs (for protocols, see Woods and Tilly, Nature Protocols, 8:966-88 (2013)) isolated from ovaries 3-month-old and 20-month-old female mice of FIG. 1A, confirming that OSCs from aged females are still capable of oocyte formation despite the fact that their ovaries lack oocytes.

FIGS. 2A-D are graphs that show the OSCs of aged mice lose the ability to support primordial follicle formation. Transgenic mice, ranging from 2-11 months, having an inducible “suicide gene” (herpes simplex virus thymidine kinase or HSVtk) that specifically disrupts OSC differentiation into oocytes only in the presence of the HSVtk pro-drug ganciclovir (GCV), were tested for their ability to lose and regain their oocyte reserves after activation and deactivation of the suicide gene, respectively.

FIG. 3 is graph that shows that intraovarian transplantation of young mouse ovarian somatic cells enriched for granulosa cells increase the primordial follicle pool in recipient aged mice (i.e., 10-month old mice) that are no longer capable of using their endogenous OSCs to generate new oocytes and follicles (see FIG. 2). The left column of each pair of columns are aged mock-transplanted control mice and the right column of each pair of columns are aged mice that received a transplant of young ovarian tissue-derived cells. The columns reflective of primordial follicle numbers (the columns encircled), which represent the earliest stage of oocytes that can be newly formed, are enhanced in the center of the graph.

FIG. 4A is a chart that shows the yield of OSCs from women during both pre-menopausal (22-47 years of age) and post-menopausal (53 and 58 years of age) life, confirming that OSCs are still present in aged human ovaries.

FIG. 4B is a picture of an immature oocyte produced in vitro from cultured OSCs isolated from a post-menopausal (53 years of age) human ovarian cortical tissue fragment.

FIG. 5A is a graph showing estradiol production by FACS-purified Fox12-DsRed positive cells (2×10³ cells per well), which spontaneously differentiated in embryonic stem cell cultures, maintained in culture for up to 3 days (FSH, 100 ng/ml; 8-br-cAMP, 1 mM). Data are the mean±SEM of 3 independent cultures (*, P<0.05 versus vehicle control).

FIG. 5B is a graph showing progesterone production by FACS-purified Fox12-DsRed positive cells (2×10³ cells per well), which spontaneously differentiated in embryonic stem cell cultures, maintained in culture for up to 3 days (FSH, 100 ng/ml; 8-br-cAMP, 1 mM). Data are the mean±SEM of 3 independent cultures (*, P<0.05 versus vehicle control).

FIG. 6A is an image showing wild-type neonatal ovary before injection of Fox12-DsRed-expressing cells isolated from ESC cultures 12 days post-differentiation.

FIG. 6B is an image showing wild-type neonatal ovary after injection of Fox12-DsRed-expressing cells isolated from ESC cultures 12 days post-differentiation.

FIG. 6C is an image showing that DsRed-expressing cells are present within the ovarian stroma at 8 days post-transplant (left); by dual immunofluorescence, these cells frequently associate with immature oocytes, identified by expression of the oocyte marker Dazl (green; right panels).

FIG. 6D is an image showing that DsRed-expressing cells are found only in the granulosa cell layer of growing follicles at 14 days post-transplant.

FIG. 7A shows visualization of growing follicles (approximately 250 micrometers in diameter; arrows) by light microscopy in human ovarian cortical strips cultured ex vivo for two weeks.

FIG. 7B shows an assessment of oocytes in human ovarian cortical tissue by DDX4 immunofluorescence after 14 days of ex vivo culture, which reveals numerous primordial and primary follicles (left) and several multilaminar follicles (right).

FIG. 8A is graph depicting the rate of in vitro maturation of oocytes contained in granulosa/cumulus cell complexes to fully mature metaphase II eggs, wherein the granulosa/cumulus cell-oocyte complexes were initially harvested from immature preantral stage (<2 mm in diameter) follicles, or more mature early antral stage (>3 mm in diameter) follicles, present in adult bovine ovarian cortical fragments (the number of oocytes analyzed per group is shown over the respective bars).

FIG. 8B shows an image of a fully mature metaphase II egg, with the extruded first polar body visible (arrow), that was successfully matured entirely in-vitro from a granulosa cell-oocyte complex harvested from a follicle less than 2 mm in diameter.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the “administration” of an agent, drug, compound, or cells to a subject includes any route of introducing or delivering to a subject an agent, drug, compound, or cells to perform its intended function. Administration can be carried out by any suitable route, including, e.g., localized injection (e.g., catheter administration or direct intra-ovarian injection), systemic injection, intravenous injection, intrauterine injection, orally, intranasally, and parenteral administration. Administration includes self-administration and the administration by another.

As used herein, “differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, muscle cell or granulosa cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). Oocytes are an example of a terminally differentiated cell type.

As used herein, the term “effective amount” or “therapeutically effective amount” refers to a quantity suitable to achieve a desired effect, e.g., an amount of granulosa cells, e.g., synthetic granulosa cells, that will e.g., elevated ovarian derived hormones and growth factors levels in a subject in need thereof or support differentiation of an oocyte precursor cell to an oocyte. By way of example, but not by way of limitation, in some embodiments, a therapeutically effective amount of granulosa cells is the amount of granulosa cells necessary to raise a subject's ovarian derived hormones and/or growth factors levels. In the context of hormone therapy applications, in some embodiments, the amount of granulosa cells or granulosa cell precursors administered to the subject will depend on the condition or disease state of the subject, e.g., a menopause subject or subject who has had a hysterectomy, and on the characteristics of the subject, such as general health, age, sex, body weight and tolerance to drugs. The skilled artisan will be able to determine appropriate dosages depending on these and other factors.

As used herein, the term “enriched population” refers to a purified or semi-purified population of cells, such as granulosa cells or granulosa cell precursors (e.g., synthetic granulosa cells). In some embodiments, a specific population of granulosa cells or granulosa cell precursors is enriched by sorting the granulosa cells or granulosa cell precursors from the population of differentiating multi-potent cells, e.g., by fluorescence activated cell sorting (FACS), magnetic assisted cell sorting (MACS), or other cell purification strategies known in the art for separation of a specific populations of cells from a general population of cells. By way of example but not by limitation, in some embodiments, an enriched population of granulosa cells or granulosa cell precursors is a purified or semi-purified population of granulosa cells or granulosa cell precursors that have been isolated from differentiating multi-potent cells by FACS.

As used herein, a “follicle” refers to an ovarian structure including a single oocyte surrounded by somatic (granulosa without or with theca-interstitial) cells. Each fully formed follicle is enveloped in a complete basement membrane. Although some of these newly formed follicles start to grow almost immediately, most of them remain in the resting stage until they either degenerate or some signal(s) activate(s) them to enter the growth phase.

As used herein, the term “immature oocyte” refers to primary oocytes that are arrested in prophase I.

As used herein, the term “mature follicle” refers to a follicle that has actively proliferating granulosa cells surrounding a developing oocyte that responds to exogenous hormones, and in particular gonadotropin hormones (follicle-stimulating hormone or FSH, and luteinizing hormone or LH). By way of example, but not by limitation, mature or maturing follicles increase in size due to proliferation of the granulosa cells, expansion of the oocyte following resumption of meiosis, and/or because of the development of a fluid filled antrum.

As used herein, the term “mature oocyte” (also referred to as an egg) refers to an oocyte arrested in metaphase II of meiosis capable of fertilization following sperm penetration or activation of parthenogenesis by addition of calcium ionophore.

As used herein, the term “granulosa stimulating agent” refers to any compound, hormone, peptide, drug, or other agent that stimulates granulosa cells or granulosa cell precursors to secrete ovarian derived hormones, e.g., estradiol or progesterone, and growth factors. By way of example, but not by way of limitation, in some embodiments, granulosa stimulating agents include but are not limited to follicle stimulating hormone (FSH) and 8-Bromoadenosine 3′,5′-cyclic monophosphate (8-br-cAMP).

As used herein, the terms “subject,” “individual,” or “patient” can be an individual organism, a vertebrate, a mammal, or a human.

As used herein, the term “synthetic granulosa” refers to granulosa cells and/or granulosa precursor cells that are produced at least partially in vitro from the directed differentiation of multi-potent cells.

### General

Studies have shown that mouse embryonic stem cells (ESCs) and induced pluripotent stem cells (iPSCs) can be differentiated, albeit at low frequency, into oocytes capable of fertilization, embryogenesis and birth of viable offspring. Hayashi et al., Science 338:971-975 (2012). These studies also demonstrate that primordial germ cell (PGC)-like cells (PGCLCs) that spontaneously arise in cultures of differentiating ESCs or iPSCs and which resemble endogenous primordial germ cells (PGCs) in fetal gonads, require interaction with developmentally matched embryonic ovary somatic cells to realize their full potential in vivo. In order to provide the micro-environmental cues necessary for oogenesis, folliculogenesis, and ultimately egg formation from PGCLCs, a source of developmentally matched ovarian somatic cells is required.

Follicle-like structures formed by mouse ESCs in vitro include a single oocyte-like cell, which can grow as large as 70 μm diameter, surrounded by one or more layers of tightly-adherent somatic cells that resemble to some degree ovarian granulosa cells. Hubner et al., Science 300:1251-1256 (2003). Analogous to what is observed during normal follicle formation within the ovary, somatic cells within ESC-derived follicle-like structures are connected via intercellular bridges with their enclosed germ cells, which may serve to facilitate cell-to-cell interaction required for normal follicle development. Additionally, increased expression of steroidogenic pathway genes, along with estrogen secretion into the culture medium, occurs concomitant with the formation of follicle-like structures from ESCs in vitro. While these observations collectively support the notion that somatic cells of in vitro-derived follicle-like structures have features ultra-structurally and functionally similar to endogenous granulosa cells, isolation and characterization of these cells from differentiating ESCs has been difficult.

Ovarian failure and the resulting menopause occur due to a loss of ovarian follicles, which are the primary endocrine producing structures in the ovaries. With this loss of follicles and the steroid producing ovarian granulosa cells comes a diminished ability to produce steroid hormones, resulting in a profound detrimental effect on women's health, impacting not only reproductive organs and tissues but bone, brain, and the cardiovascular system. The net result is a decline in bone density and cognitive function with age, as well as an increase in cardiovascular diseases (CVDs), which are the leading causes of death in women worldwide. Currently, menopausal hormone therapy (MHT; previously referred to as hormone replacement therapy, or HRT) is used to temporarily offset some of the symptoms that accompany menopause, but MHT comes with a number of well-documented caveats and health risks. Accordingly, strategies to generate steroid producing ovarian granulosa cells from stem cells that could work in concert with the hypothalamic gonadal axis could fill a critical void in the current management of ovarian failure and menopause.

Attempts to recapitulate an ovarian-like environment in vitro have been published. Using a 3-dimensional (3-D) in vitro maturation (IVM) culture system, it has been demonstrated that combining the three follicular subtypes (e.g., theca, granulosa, and oocytes) creates an ‘artificial’ ovarian- or follicle-like environment which supports human oocyte maturation. Similar strategies for follicle culture have been reported in mice, rats and primates, with ex vivo follicle development leading to oocyte maturation. The potential utility in MHT, however, has only recently been explored. Drawing from previous work on ovarian follicle cultures using a 3-D alginate encapsulation, some data indicate that multilayered co-cultures of theca and granulosa cells obtained from mouse ovaries can be sustained in vitro for at least a month. During this timeframe, the encapsulated co-cultures functioned in a similar capacity to that of native follicles demonstrated by the synthesis of estradiol and progesterone, and secretion of inhibin in following gonadotropin stimulation. Given that the most obvious drawback to MHT is a lack of communication between all components of the hypothalamo-pituitary-gonadal (HPG) axis, a cell or tissue based strategy to promote endocrine function has the real potential to circumvent this issue. However, the source of the cells that can be used for such a therapy is currently limited, as patients requiring such a treatment have few to no granulosa or theca cells.

The present technology provides an improved method for recapitulating an artificial ovarian environment by using a multi-potent cell-based method that produces granulosa and/or granulosa precursor cells. In general, the present technology relates to methods for the directed differentiation of multi-potent cells into granulosa and/or granulosa precursor cells. Additionally, the present technology relates to the use of the granulosa and/or granulosa precursor cells produced by the directed differentiation of the multi-potent cells.

Methods for the Directed Differentiation of Multi-potent Cells into Granulosa Cells and/or Granulosa Precursor Cells

In some embodiments, methods for the directed differentiation of multi-potent cells into granulosa and/or granulosa precursor cells (hereinafter “synthetic granulosa cells”) includes culturing multi-potent cells in conditions suitable for differentiation of the multi-potent cells to synthetic granulosa cells.

In some embodiments, the conditions suitable for differentiation of the multi-potent cells to synthetic granulosa cells includes, but is not limited to, separating the multi-potent cells (e.g., embryonic stem cells) from a mitotically-inactivated mouse embryonic fibroblast (MEF) feeder layer by differential adhesion and culturing multi-potent cells the absence of leukemia inhibitory factor (LIF). In some embodiments, the multi-potent cells are plated on gelatin-coated plates in a monolayer after removal from the MEF feeder layer. In some embodiments, the multi-potent cells are cultured with 15% FBS in the absence of LIF.

Additionally, or alternatively, in some embodiments, a suitable condition for differentiation of the multi-potent cells to synthetic granulosa cells includes, but is not limited to, contacting the multi-potent cells with mesoderm-specifying agents such as a glycogen synthase kinase-3 (GSK-3) inhibitor, bone morphogenetic protein (BMP4; 1-1,000 ng/ml), retinoic acid (RA; 0.001-10 μM), or a combination thereof.

By way of example, but not by way of limitation, in some embodiments, GSK-3 inhibitors include, but are not limited to, SB216763 (1-20 μM), BIO (0.1-10 μM), CHIR99021 (0.1-10 μM), lithium chloride (LiCl), maleimide derivatives, staurosporine, indole derivatives, paullone derivatives, pyrimidine and furopyrimidine derivatives, oxadiazole derivatives, thiazole derivatives, and heterocyclic derivatives.

In some embodiments, the multi-potent cells are contacted with growth factors or activators of signaling pathways for granulosa cell specification to direct multi-potent cells to differentiate into synthetic granulosa cells. Growth factors or activators of signaling pathways for granulosa cell specification, include, but are not limited to bFGF or activators of the Notch signaling pathway, e.g., Jagged1 or Jagged2.

In some embodiments, the method for the directed differentiation of multi-potent cells to synthetic granulosa cells is a stepwise method comprising:

Step 1) culturing multi-potent cells in a monolayer in absence of MEFs and LIF and in the presence of at least one GSK-3 inhibitor; and

Step 2) adding BMP4 and/or RA to the culture medium.

In some embodiments, the multi-potent cells are cultured in Step 1 for between about 1 hour to 48 hours, about 4 hours to 44 hours, about 8 hours to 40 hours, about 12 hours to 36 hours, about 16 hour to 32 hours, about 20 hours to 28 hours, or about 22 hours to 26 hours. In some embodiments, the multi-potent cells are cultured in Step 1 for about 24 hours.

In some embodiments, the multi-potent cells are incubated with BMP4 and/or RA in Step 2 for between about 1 hour to 48 hours, about 4 hours to 44 hours, about 8 hours to 40 hours, about 12 hours to 36 hours, about 16 hour to 32 hours, about 20 hours to 28 hours, or about 22 hours to 26 hours. In some embodiments, the multi-potent cells are incubated with BMP4 and/or RA in Step 2 for about 24 hours.

In some embodiments, the multi-potent cells are engineered to express one or more genes that specify granulosa cells and/or granulosa cell precursors. In some embodiments, the gene or genes is/are inducible. In some embodiments, induction of the gene or genes that specify granulosa cells and/or granulosa cell precursors directs differentiation of the multi-potent cells to synthetic granulosa cells.

By way of example, but not by way of limitation, in some embodiments, genes that specify (e.g., are biomoarkers for and/or elicit differentiation to) granulosa cells and/or granulosa cell precursors include, but are not limited to, forkhead box L2 (Fox12), wingless type MMTV integration site family, member 4 (WNT4), Nr5a1, Dax-1, ATP-binding cassette, subfamily 9 (Abca9), acetyl-Coenzyme A acyltransferase 2 (mitochondrial 3-oxoacyl-Coenzyme A thiolase; Acaa2), actin, alpha 2, smooth muscle, aorta (Acta2), a disintegrin-like and metallopeptidase (reprolysin-like) with thrombosin type 1 motif, 17 (Adamts17), ADAMTS-like 2 (Adamts12), AF4/FMR2 family, member 1 (Aff1), expressed sequence AI314831 (AI314831), Aldo-keto reductase family 1, member C14 (Akr1c14), aldo-keto reductase family 1, Notch2, and member C-like (Akr1c1).

Engineering multi-potent cells to contain one or more genes that specify granulosa cells and/or granulosa cell precursors can be accomplished by any method known in the art. By way of example, but not by limitation, in some embodiments, the one or more genes that specify granulosa cells and/or granulosa cell precursors are inserted into the multi-potent cells by using a technique selected from the group consisting of electroporation, viral transduction, cationic liposomal transfection, multi-component lipid based transfection, calcium phosphate, DEAE-dextran, and direct delivery.

In some embodiments, multi-potent cells are engineered to contain at least one granulosa cell specific gene reporter, wherein expression of the granulosa cell specific gene reporter is indicative of a cell that is a granulosa cell or a granulosa cell precursor.

In some embodiments, the granulosa cell specific reporter includes a fluorescent reporter under regulatory control of a granulosa cell-specific gene. In some embodiments, the granulosa cell-specific gene that controls the granulosa cell specific report is the same gene that is inducibly expressed in the multi-potent cells.

Ovarian granulosa cell-specific genes include, but are not limited to, forkhead box L2 (Fox12), wingless type MMTV integration site family, member 4 (WNT4), Nr5a1, Dax-1, ATP-binding cassette, subfamily 9 (Abca9), acetyl-Coenzyme A acyltransferase 2 (mitochondrial 3-oxoacyl-Coenzyme A thiolase; Acaa2), actin, alpha 2, smooth muscle, aorta (Acta2), a disintegrin-like and metallopeptidase (reprolysin-like) with thrombosin type 1 motif, 17 (Adamts17), ADAMTS-like 2 (Adamts12), AF4/FMR2 family, member 1 (Aff1), expressed sequence AI314831 (AI314831), Aldo-keto reductase family 1, member C14 (Akr1c14), aldo-keto reductase family 1, Notch2, and member C-like (Akr1c1).

Fluorescent reporters include, but are not limited to, Discosoma sp. red (DsRed), green fluorescent protein (GFP), yellow fluorescent protein (YFP), and orange fluorescent protein (OFP).

In some embodiments, the granulosa cell specific reporter is a non-fluorescent reporter under regulatory control of a granulosa cell-specific gene. Non-fluorescent reporters include, but are not limited to, luciferase and beta-galactosidase.

The granulosa cell specific reporter can be engineered by any methods known in the art. By way of example, but not by limitation, in some embodiments, a granulosa cell specific reporter is engineered by identifying a granulosa cell specific gene promoter, determining a conserved region of the gene promoter, isolating the conserved region from genomic DNA using PCR, and cloning the conserved region into a vector containing a fluorescent marker.

Engineering multi-potent cells to contain the granulosa cell specific gene reporter can be accomplished by any method known in the art. By way of example, but not by limitation, in some embodiments, the granulosa cell specific gene reporter are inserted into the multi-potent cells by using a technique selected from the group consisting of electroporation, viral transduction, cationic liposomal transfection, multi-component lipid based transfection, calcium phosphate, DEAE-dextran, and direct delivery.

In some embodiments, the method for directed differentiation of multi-potent cells into synthetic granulosa cells includes a combination of any one of the above described suitable culture conditions and above described engineered multi-potent cells. By way of example, but not by way of limitation, in some embodiments, the method for directed differentiation of multi-potent cells into synthetic granulosa cells includes culturing multi-potent cells in culture conditions that include the absence of MEFs and LIF and the presence of a GSK inhibitor, wherein the multi-potent cells are engineered to express one or more genes that specify granulosa cells and/or granulosa cell precursors and inducing expression of the one or more genes that specify granulosa cells and/or granulosa cell precursors, and thereby leading to the formation of synthetic granulosa cells.

In some embodiments, after inducement of differentiation of the population of multi-potent cells, synthetic granulosa cells are identified and isolated. In some embodiments, the synthetic granulosa cells are identified by the expression of a fluorescent marker under the control of a granulosa cell-specific gene. In some embodiments, the synthetic granulosa cells are isolated by forming enriched populations of synthetic granulosa cells precursors by FACS, antibody-based immunomagnetic sorting (e.g., magnetic assisted cell sorting (MACS)), differential adhesion, clonal selection and expansion, or antibiotic resistance.

In some embodiments, the synthetic granulosa cells are isolated using a cell surface marker(s) selective for or specific to granulosa cells or granulosa cell precursors. Examples of cell surface markers selective for or specific to granulosa cells or granulosa cell precursors include, but are not limited to anti-Müllerian hormone receptor, and Notch receptor (Notch2).

In some embodiments, the multi-potent cells include, but are not limited to, embryonic stem cells (ESCs), pluripotent stem cells, very small embryonic-like (VSEL) cells, induced pluripotent stem cells (iPSCs) or otherwise reprogrammed somatic cells, skin cells, bone marrow derived cells, and peripheral blood-derived cells.

The multi-potent cells may be any mammalian multi-potent cell. Mammals from which the multi-potent cell can originate, include, for example, farm animals, such as sheep, pigs, cows, and horses; pet animals, such as dogs and cats; laboratory animals, such as rats, mice, monkeys, and rabbits. In some embodiments, the mammal is a human.

### Methods for Growth and Maturation of Follicles and Immature Oocytes in Ovarian Tissue

In some embodiments, the synthetic granulosa cells (i.e., granulosa cells and/or granulosa cell precursors produced by the methods above) are used to promote the growth and maturation of follicles, follicle-like structures, and/or oocytes in ovarian tissue.

In some embodiments, ovarian tissue is contacted with a population of synthetic granulosa cells, wherein the synthetic granulosa cells promote the growth and maturation of follicles, follicle-like structures, and/or immature oocytes in ovarian tissue. In some embodiments, after contact with the ovarian tissue, the synthetic granulosa cells migrate to follicles, follicle-like structures, and/or immature oocytes or oocyte precursor cells in ovarian tissue to produce an ovarian somatic environment that induces maturation of follicles and/or oocytes.

In some embodiments, the ovarian tissue is contacted with the synthetic granulosa cells in vivo. In some embodiments, in vivo administration includes, but is not limited to, localized injection (e.g., catheter administration or direct intra-ovarian injection), systemic injection, intravenous injection, intrauterine injection, and parenteral administration. In some embodiments, the synthetic granulosa is administered to a subject in need thereof.

By way of example, but not by way of limitation, in some embodiments, a subject in need thereof is a subject that is having trouble conceiving, undergoing infertility treatment, undergoing in vitro fertilization, been treated for cancer, has been subjected to cytotoxic therapies (e.g., chemotherapy or radiotherapy), or a combination thereof.

In some embodiments, the ovarian tissue is contacted by the synthetic granulosa cells ex vivo. In some embodiments, ex vivo contact includes, but is not limited to aggregation with intact or dissociated removed ovarian tissue, and co-culture with ovarian tissue. In some embodiments, the contacted ex vivo ovarian tissue is cultured and then transplanted or implanted into a subject's ovaries or surrounding tissues. Methods for transplanting or implanting include, but are not limited to, engraftment onto ovary, injection or engraftment of tissue into ovary following ovarian incision, and engraftment into fallopian tube.

In some embodiments, the ovarian tissue contacted ex vivo by the synthetic granulosa cells is frozen and stored, e.g., after growth and maturation of the follicle and/or oocyte.

The ovarian tissue may be any mammalian ovarian tissue. Mammals from which the ovarian tissue can originate, include, for example, farm animals, such as sheep, pigs, cows, and horses; pet animals, such as dogs and cats; laboratory animals, such as rats, mice, monkeys, and rabbits. In some embodiments, the mammal is a human.

In some embodiments, the synthetic granulosa cells and the ovarian tissue are autologous (from the same individual). In some embodiments, the synthetic granulosa cells and the ovarian tissue are heterologous (allogeneic, from different individuals).

In some embodiments, the promotion of growth and maturation of follicles, follicle-like structures, and/or immature oocytes or oocyte precursors in ovarian tissue by the synthetic granulosa cells is measured by an increase in follicle diameter, increase in granulosa cell number, increase in steroid hormone production, increase in oocyte diameter, or a combination thereof.

The diameter of a maturing follicle or oocyte varies from species to species and is identifiable by one skilled in the art since mature follicle sizes for specific species is generally known in the art. By way of example, but not by limitation, in some embodiments, a follicular diameter of a human follicle that is indicative of a mature or maturing follicle is a diameter greater than about 30 μm. Alternatively, or additionally, a follicular diameter of a human follicle that is indicative of a mature or maturing follicle is a diameter between about 30 μm to 10,000 μm, between about 50 μm to 5000 μm, between about 100 μm to 2000 μm, between about 200 μm to 1000 μm, between about 300 μm to 900 μm, between about 400 μm to 800 μm, or between about 500 μm to 700 μm.

By way of example, but not by limitation, in some embodiments, an oocyte diameter of a human oocyte that is indicative of a mature or maturing oocyte is a diameter greater than about 10 μm. Alternatively, or additionally, a diameter of an oocyte contained in a human follicle that is indicative of a mature or maturing oocyte is a diameter between about 10 μm to 200 μm, or between about 20 μm to 175 μm, or between about 30 μm to 150 μm, or between about 40 μm to 125 μm, or between about 50 μm to 100 μm, or between about 60 μm to 75 μm.

In some embodiments, an increase in granulosa cell number in ovarian tissue is measured by comparison of the number of granulosa cells in the ovarian tissue before contact with the synthetic granulosa cells to the number of granulosa cells in the ovarian tissue after contact with the synthetic granulosa cells. Alternatively, or additionally, an increase in granulosa cell number in ovarian tissue is measured by comparison of the number of granulosa cells in the ovarian tissue after contact with the synthetic granulosa cells as compared to age-matched ovarian tissue not contacted with the synthetic granulosa cells.

In some embodiments, the increase in granulosa cell number in ovarian tissue contacted with synthetic granulosa cells is measured as a percent increase of about 1%, 5%, 10%, 20%, 30%, 40%, 50%, 60%, 70%, 80%, 90%, 100% or a percent increase between any two of these values as compared to, e.g., ovarian tissue before contact with synthetic granulosa cells or age-matched ovarian tissue not contacted with synthetic granulosa cells.

Steroid hormones produced by the contacting of the synthetic granulosa cells with ovarian tissue include, but are not limited to, estradiol, estriol, estrone, pregnenolone, and progesterone. In some embodiments, the increase in steroid hormones produced in ovarian tissue contacted with the synthetic granulosa cells is measured as a percent increase of about 1%, 5%, 10%, 20%, 30%, 40%, 50%, 60%, 70%, 80%, 90%, 100% or a percent increase between any two of these values as compared to, e.g., the ovarian tissue before contact with the synthetic granulosa cells or age-matched ovarian tissue not contacted with the synthetic granulosa cells.

### Ex Vivo and In Vivo Systems and Methods for Generating Mature Follicle Containing a Mature Oocyte

### Ex Vivo and In Vivo Systems

In some embodiments, a system for producing an ex vivo or in vivo artificial ovarian environment that produces a mature follicle containing a mature oocyte includes synthetic granulosa cells (i.e., any one of the granulosa cell and/or granulosa precursor cells engineered from directed differentiation of multi-potent cells described above), oocyte precursor cells, and ovarian tissue. In some embodiments, the synthetic granulosa cells, the oocyte precursor cells, and the ovarian tissue are autologous. In some embodiments, the synthetic granulosa cells, the oocyte precursor cells, and the ovarian tissue are heterologous allogeneic.

In some embodiments, the oocyte precursor cells are engineered from multi-potent cells or oocyte-producing germ line cells. In some embodiments, the multi-potent cells to be used for production of oocyte precursor cells or oocytes include, but are not limited to, embryonic stem cells (ESCs), pluripotent stem cells, induced pluripotent stem cells (iPSCs) or otherwise reprogrammed somatic cells, very small embryonic like (VSEL) cells, skin cells, bone marrow derived cells, and peripheral blood-derived cells. In some embodiments, the oocyte-producing germ line cells include, but are not limited to, primordial germ cells, female germ line stem cells (fGSCs) or oogonial stem cells (OSCs). Engineering oocyte precursors from multi-potent cells or oocyte-producing germ line cells can be performed using any method commonly known in the art. See, e.g., Hayashi et al., Science, 338: 971-975 (2012); White et al., Nature Medicine 2012 18: 413-421 (2012).

In some embodiments, the oocytes precursor cells contain at least one genetic modification. In some embodiments, the genetic modification occurs in the multi-potent cells. In another embodiment, the genetic modification occurs in the oocyte-producing germ line cells. Without wishing to be bound by theory, genetic modifications in the multi-potent cells or oocyte-producing germ line cells are maintained throughout differentiation, thus the resulting is an oocyte precursor, and/or ultimately an oocyte, that is a carrier of the genetic modification. In yet another embodiment, the genetic modification occurs in the oocyte-precursor cells.

Genetic modification of the multi-potent cells, oocyte-producing germ line cells, or oocyte-precursor cells can be performed by one or more techniques commonly used in the art. By way of example, but not by way of limitation, gene modification techniques include, but are not limited to, electroporation, direct injection of encoding mRNAs, lipid based transfection, retroviral transduction, adenoviral transduction, lentiviral transduction, CRISPR/Cas9, TALENs, zinc finger nucleases (ZFNs), engineered meganucleases, and site directed mutagenesis. See, e.g., Shao et al., Nature Protocols, 9(10): 2493-2512 (Sep. 25, 2014), Kato et al., Scientific Reports (Nov. 5, 2013), and Yang et al., Nature Protocols, 9(8): 1956-1968 (Jul. 24, 2014).

In some embodiments, the genetic modification results in the restoration of expression of one or more missing genes (or gene products) whose expression is reduced or absent due to genetic or epigenetic changes and/or to correct existing gene mutations or deletions. In some embodiments, the missing gene or reduced or absent gene, or the gene with a mutation or deletion, leads to impaired or otherwise negatively impacts one or more events associated with fertility outcomes including, but not limited to, fertilization, embryo formation, embryo development, embryo implantation, embryo gestation to term, and/or birth of offspring free of gene mutations (e.g., loss or gain of function) responsible for onset of or susceptibility to diseases and disorders. In some embodiments, the genetic modification results in the expression of a desired gene.

In some embodiments, the artificial ovarian environment system is formed and maintained ex vivo. In some embodiments, the artificial ovarian environment system is formed and maintained in vivo.

### Methods for Making Mature Follicle and Mature Oocytes in an Ex Vivo or In Vivo System

In some embodiments, an ex vivo artificial ovarian environment is made by combining synthetic granulosa cells (made by any one of the methods described above), oocyte precursor cells (made by any one of the methods described above), and ovarian tissue in conditions suitable to produce a mature follicle and mature oocyte. Any known methods and suitable conditions for making ex vivo artificial ovarian environments or for the maturation of immature follicles and oocytes to mature follicles and oocytes can be used. See, e.g., Shea and Woodruff, WO 2007/075796; Albertini and Akkoyunlu, Methods in Enzymology 426:107-121 (2010); Jin et al., Fertil Steril 93:2633-2639 (2010); White et al., Nature Medicine 18:413-421 (2012); Telfer and MacLaughlin, Int J Dev Biol 56:901-907 (2012).

In some embodiments, the conditions suitable to produce a mature follicle and mature oocyte include the presence of growth factors. Growth factors that are useful to produce mature follicle and mature oocyte include, but are not limited to, inhibins, activins, GDF9, BMP15, IGF-1, insulin, selenites, and transferrins.

Additionally, or alternatively, in some embodiments, the conditions suitable to produce a mature follicle and mature oocyte include the presence of hormones. Hormones that are useful to produce mature follicle and mature oocyte include, but are not limited to, follicle stimulating hormone (FSH) and luteinizing hormone (LH).

In some embodiments, a mature follicle and/or a mature oocyte produced in the ex vivo artificial ovarian environment is injected, transferred or otherwise delivered back into a subject.

In some embodiments, a mature oocyte produced in the ex vivo artificial ovarian environment is subjected to in vitro fertilization. In some embodiments, the in vitro fertilized mature oocyte produced in an ex vivo artificial ovarian environment of the present technology is injected, transferred or otherwise delivered back into a subject.

In some embodiments, a mature follicle and/or a mature oocyte produced in the ex vivo artificial ovarian environment is frozen for future use. In some embodiments, the in vitro fertilized mature oocyte produced in an ex vivo artificial ovarian environment of the present technology is frozen for future use.

In some embodiments, an in vivo artificial ovarian environment is made by injecting synthetic granulosa cells (made by any one of the methods described above) and oocyte precursor cells (made by any one of the methods described above) into the ovarian tissue of a subject.

In some embodiments, the subject is a mammal Mammalian subjects, include, but are not limited to, farm animals, such as sheep, pigs, cows, and horses; pet animals, such as dogs and cats; laboratory animals, such as rats, mice, monkeys, and rabbits. In some embodiments, the mammal is a human.

In some embodiments, the use of mature follicles and/or mature oocytes developed in the ex vivo or in vivo system described above is useful for improving fertility.

In some embodiments, the use of mature follicles and/or mature oocytes developed in the ex vivo or in vivo system described above is useful for reducing the inheritance of genetic diseases and/or disorders and/or for reducing the prevalence of carriers of a disease or disorder.

In some embodiments, the use of mature follicles and/or mature oocytes developed in the ex vivo or in vivo system described above is useful as an option for female subjects undergoing in vitro fertilization.

In some embodiments, the use of mature follicles and/or mature oocytes developed in the ex vivo or in vivo system described above is useful as an option for improved in vitro fertilization for female subjects treated for cancer or subjected to cytotoxic therapies, e.g., chemotherapy, radiation therapy, or both.

In some embodiments, genetically modified oocyte precursor cells (as described above) are combined only with ovarian tissues and cultured ex vivo in conditions suitable to produce mature follicles and/or mature oocytes. In some embodiments, the mature oocyte is frozen for later use, e.g., IVF. In some embodiments, the mature oocyte no longer carries the genetic defect or expresses a desired gene.

Methods for Increasing Ovarian-derived Hormones and Growth Factors in a Subject

In some embodiments, an effective amount of the synthetic granulosa cells (i.e., any one of the granulosa cell and/or granulosa precursor cells engineered from directed differentiation of multi-potent cells described above) is administered to a subject to increase ovarian-derived hormones and growth factors.

In some embodiments, the synthetic granulosa cells secrete ovarian-derived hormones and growth factors. Alternatively, or additionally, in some embodiments, the synthetic granulosa cells are stimulated to secrete ovarian-derived hormones and growth factors by one or more granulosa stimulating agents.

Ovarian-derived hormones secreted by the synthetic granulosa cells include, but are not limited to, estradiol, estriol, estrone, pregnenolone, and progesterone. Ovarian-derived growth factors secreted by the synthetic granulosa cells include, but are not limited to, activin and inhibin.

In some embodiments, the synthetic granulosa cells are stimulated before administration to the subject, i.e., the synthetic granulosa cells are stimulated ex vivo to secrete ovarian derived hormones and growth factors. In some embodiments, the synthetic granulosa cells are stimulated after administration to the subject, i.e., the synthetic granulosa cells are stimulated in vivo to secrete ovarian-derived hormones and growth factors.

Granulosa stimulating agents include, but are not limited to, follicle-stimulating hormone (FSH), 8-Bromoadenosine 3′,5′-cyclic monophosphate (8-br-cAMP), and luteinizing hormone (LH).

In some embodiments, the synthetic granulosa cells are autologous to the subject (e.g., were derived from the subject's own multi-potent cells). In some embodiments, the synthetic granulosa cells are heterologous to the subject (e.g., were derived from the multi-potent cells of another individual).

In some embodiments, the subject suffers from reduced or lack of secretion of ovarian-derived hormones and growth factors. In some embodiments, the reduced or lack of secretion of ovarian-derived hormones and growth factors is due to menopause, ovariectomy, hysterectomy, premature ovarian failure, primary ovarian insufficiency, chemotherapy-induced ovarian failure, and/or Turner's syndrome.

In some embodiments, an increase in ovarian-derived hormones and growth factors in a subject in need thereof is based on a comparison between ovarian-derived hormones and growth factors levels in the subject before administration of the synthetic granulosa cells to ovarian-derived hormones and growth factors levels in the subject after administration of the synthetic granulosa cells.

In some embodiments, an increase in ovarian-derived hormones and growth factors in a subject is based on the ovarian-derived hormones and growth factors levels in a subject after administration of synthetic granulosa cells as compared to ovarian-derived hormones and growth factors levels in a subject, who is sex and aged matched to the treated subject and not administered granulosa cells or granulosa cell precursors.

In some embodiments, the increase in ovarian-derived hormones and growth factors produced in a subject administered granulosa cells or granulosa cell precursors is measured as a percent increase of about 1%, 5%, 10%, 20%, 30%, 40%, 50%, 60%, 70%, 80%, 90%, 100% or a percent increase between any two of these values as compared to, e.g., the subject before contacting with synthetic granulosa cells or a sex and aged matched subject not administered synthetic granulosa cells.

The effective amount of synthetic granulosa cells may be determined during pre-clinical trials and clinical trials by methods familiar to physicians and clinicians. An effective amount of synthetic granulosa cells useful in the methods may be administered to a subject in need thereof by any of a number of well-known methods for administering cells. The dose and/or dosage regimen will depend upon the characteristics of the condition being treated, e.g., the subject is in menopause or the subject had a hysterectomy, the subject, and the subject's history.

Any method known to those in the art for administration of cells as a therapy may be employed. In some embodiments, the synthetic granulosa cells are administered to the subject, e.g., localized injection (e.g., catheter administration or direct intra-ovarian injection), systemic injection, intravenous injection, intrauterine injection, and parenteral administration. By way of example, but not by limitation, in some embodiments, synthetic granulosa cells precursors are directly injected into ovarian tissue or ovaries.

In some embodiments, the subject is a mammal Mammalian subjects, include, but are not limited to, farm animals, such as sheep, pigs, cows, and horses; pet animals, such as dogs and cats; laboratory animals, such as rats, mice, monkeys, and rabbits. In some embodiments, the mammal is a human.

### EXAMPLES

The present examples are non-limiting implementations of the use of the present technology.

### Example 1

### Oogonial Stem Cells (OSCs) Remain in Ovaries of Mice at Advanced Ages

This example shows that oogonial stem cells, one source of oocyte precursor cells of the invention, remain in ovaries of mice at advanced ages (i.e., 10 months or older).

### Materials and Methods

Ovary dissociation and preparation for flow cytometry. Woods and Tilly, Nature Protocols 8:966-988 (2013). C57B1/6 mice were euthanized and ovaries removed (4 ovaries per age group) and placed into 2 ml of 800 U/ml collagenase type IV in a glass dissecting dish. Ovaries were finely minced in the collagenase solution and placed at 37° C. for 15 minutes with gentle consistent agitation to generate a single cell suspension. The single cell suspension was washed with Hank's buffered saline solution (HBSS) followed by centrifugation (300×g) for 5 minutes. The supernatant was discarded and the cell pellet was re-suspended in blocking solution consisting of HBSS supplemented with normal goat serum and bovine serum albumen. The cell suspension was incubated in the blocking solution for 30 minutes. After blocking, rabbit anti-DDX4 antibody (a germ cell linage specific antibody) was added to the cell suspension, and the cells were incubated with the antibody for 30 minutes. The cell suspension was then washed with HBSS followed by centrifugation (300×g) for 5 minutes. The cells were then incubated with a fluorescent-conjugated (such as allophycocyanin (APC) or fluorescein isothiocyanate (FITC)) goat anti-rabbit secondary antibody in preparation for flow cytometry. The cell suspension was washed HBSS followed by centrifugation (300×g) for 5 minutes to remove excess fluorescent-conjugated secondary antibody. The labeled cell suspension was loaded onto a flow cytometer, and the DDX4-positive fraction (OSCs) was determined by fluorescence. The positive events were recorded, and expressed as % yield of the total viable cell population.

Culture conditions for OSCs. Woods and Tilly, Nature Protocols 8:966-988 (2013). The DDX4-positive cell fraction was collected and placed into culture conditions favorable for oogonial stem cell growth, including a mouse embryonic fibroblast (MEF) feeder layer and growth medium supplemented with 10% fetal bovine serum (FBS), 1 mM sodium pyruvate, 1 mM non-essential amino acids, 1×-concentrated antibiotic solution, 0.1 mM β-mercaptoethanol, 1×-concentrated N-2 supplement, 10³ units/ml LIF, 10 ng/ml epidermal growth factor, 1 ng/ml basic fibroblast growth factor (bFGF) and 40 ng/ml glial cell-derived neurotrophic factor (GDNF). Spontaneous differentiation of OSCs into immature oocytes was monitored by collecting culture supernatants and microscopy-based detection of oocytes. Woods and Tilly, Nature Protocols 8:966-988 (2013).

### Results

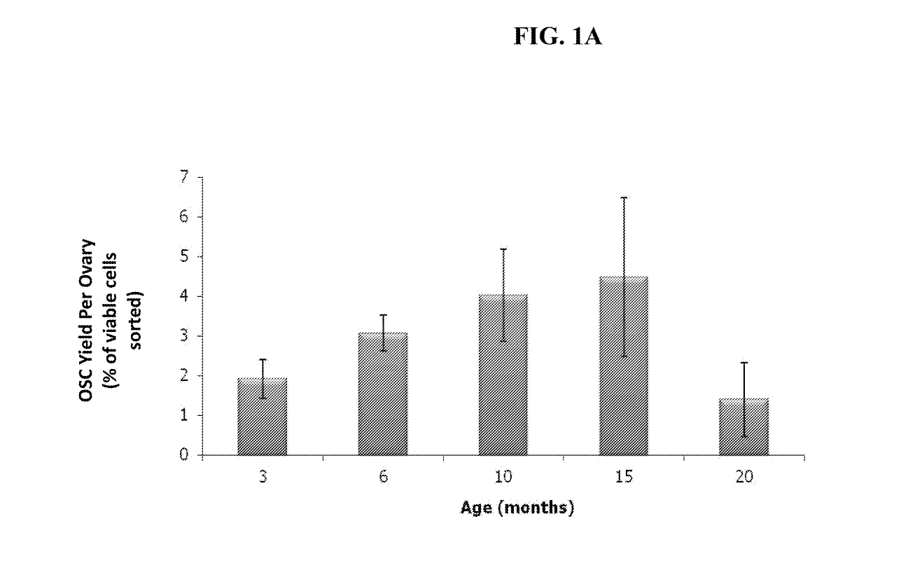

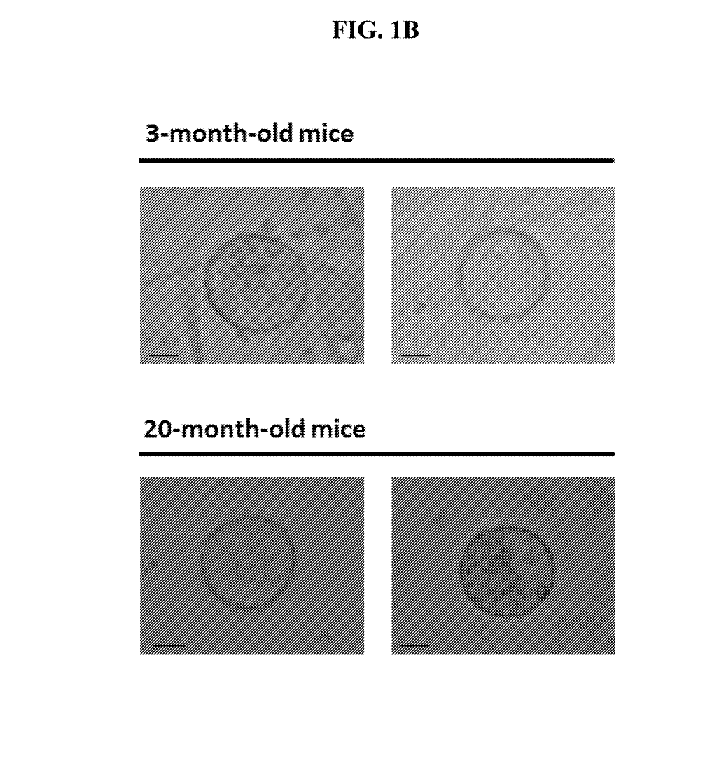

As shown in FIG. 1A, oogonial stem cells (OSCs) persist in ovaries of mice at advanced ages, even after the oocyte-containing follicle pool is completely depleted at 20 months of age. FIG. 1B shows that OSCs from aged females retain the ability to form immature oocytes (similar to OSCs from young females), once removed from the ovary tissue and cultured ex vivo. These results show that oogonial stem cells persist in advanced aged mice and that the cells from aged mice can still form immature oocytes.

### Example 2

### Oogonial Stem Cells (OSCs) Differentiation into Oocytes is Reduced in Advanced Aged Mice

This example shows that OSCs in advanced aged mice can no longer contribute to new oocyte and follicle formation.

### Materials and Methods

Animals and treatments. Transgenic pStra8-Gfp mice with expression of green fluorescent protein (GFP) driven by the promoter of the meiosis commitment gene, stimulated by retinoic acid gene 8 (Stra8;) mice were generated as described in Imudia et al., Fertil Steril, 100:1451-1458 (2013). Transgenic mice with herpes simplex virus thymidine kinase (HSVtk) expression driven by the Stra8 promoter were generated by replacing the GFP-coding sequence in the pStra8-Gfp construct with cDNA encoding GFP-fused HSVtk and the constructs were then sent to Genoway for generation of the transgenic lines, as described (Imudia et al., Fertil Steril, 100:1451-1458 (2013). For comparative studies, wild type and transgenic siblings from breeding colonies were used in parallel to rule out any potential effect of background strain on the outcomes. For treatments, the HSVtk pro-drug, ganciclovir (GCV; Roche), was dissolved in sterile water at 10 mg/ml, and then diluted in sterile 1×-concentrated phosphate-buffered saline (PBS) for daily dosing (10 mg/kg for 21 days, intraperitoneal injection). Control animals were injected with vehicle (PBS) in parallel.

Oocyte counts. Prior to the start of PBS or GCV injections, 21 days after daily dosing with GCV, and 21 days after ceasing GCV treatment, ovaries were collected from mice at the indicated ages, fixed, embedded in paraffin, and serially sectioned for histomorphometry-based quantification of the number of oocyte-containing primordial follicles, as detailed (Jones and Krohn, J Endocrinol, 21:469-495 (1961); Johnson et al., Nature, 428:145-150 (2004); and Wang and Tilly, Cell Cycle, 9:339-349 (2010)). All samples were assessed in a completely blinded fashion, and reproducibility was independently confirmed with randomly selected slides by a second observer. In all cases, variation in counts between observers was less than 7%.

### Results

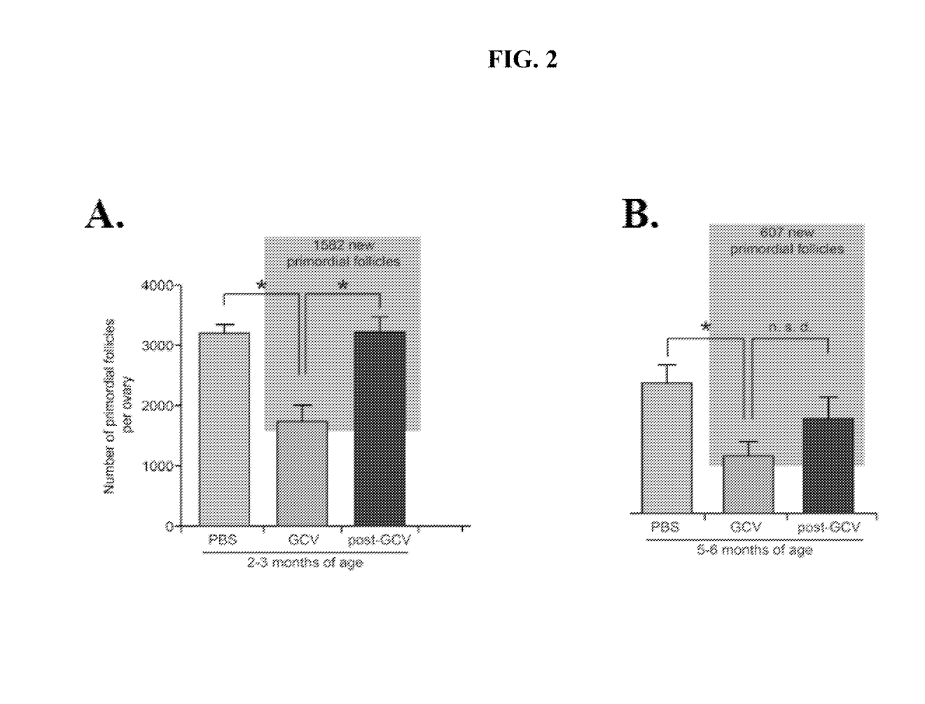

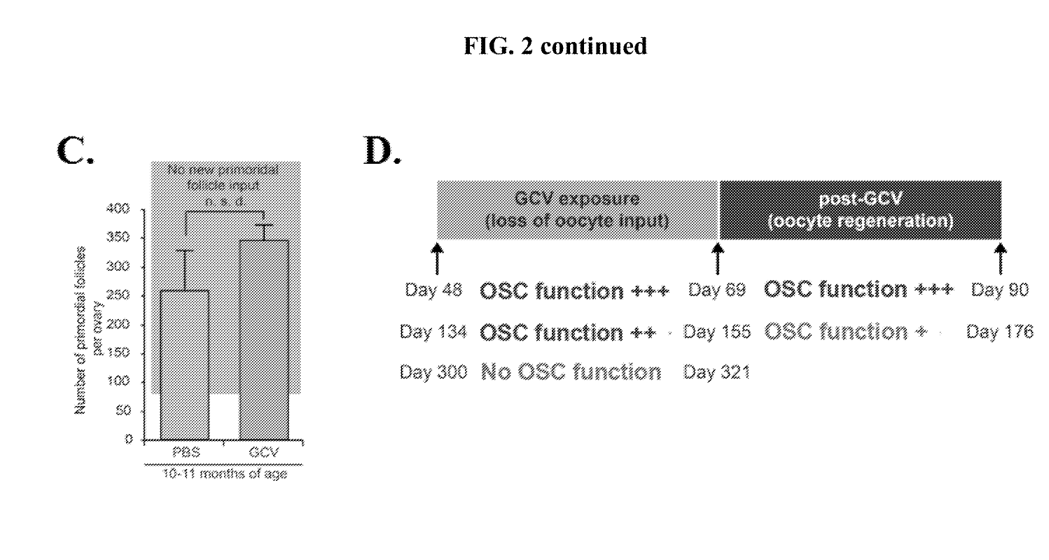

As shown in FIGS. 2A-B and 2D, young adult mice, i.e., 2-3 months of age, and middle-age adult mice, i.e., 5-6 months of age, the temporal disruption of OSC differentiation into new oocytes for 21 days by GCV treatment leads to a reduced primordial follicle reserve due to failed oocyte input. However, the primordial follicle pool regenerates back to control (PBS, vehicle) levels within 21 days of ceasing GCV treatment. The ability of GCV exposure and removal to reversibly disrupt oogenesis is progressively lost as females age (see FIGS. 2A-D). Advanced aged mice, i.e., 10-11 months of age, became completely refractory to GCV treatment (FIG. 2C, 2D), indicating that OSCs are unable to contribute any more new oocytes to the ovarian pool of follicles by this age.

In mice, the ability of OSCs to support new oocyte and follicle production is completely lost by 10-11 months of age. However, as shown in Example 1, OSCs are still present in ovaries at this age (and well beyond), indicating that aged mouse ovaries fail to provide OSCs with all of the ‘factors’ needed for new oocyte and follicle formation about halfway through chronological lifespan.

### Example 3

### Transplantation of Juvenile Mouse Ovarian Tissue-derived Cells Rescues Oocyte and Follicle Formation in Aged Mice

This example shows that dispersed ovarian tissue from juvenile mice, which is highly enriched for granulosa cells or their precursors, can support de novo follicle formation and increase the number of primordial follicles present in aged animals.

### Materials and Methods

Preparation of tissue for injection. Ovarian tissue was collected from juvenile C57B1/6 (wild type) donor mice, and dissociated into single cell suspension using collagenase type IV with gentle agitation. The dispersed ovarian tissue was washed with HBSS followed by centrifugation (300×g) for 5 minutes to remove collagenase. The cell pellet was then re-suspended and loaded into a micropipette in preparation for intraovarian injection.

Intraovarian injection. 10 month old female mice harboring a germ line-specific green fluorescent protein (GFP) transgene driven by a modified Pou5f1 (also referred to as Oct4) promoter in which the proximal enhancer has been deleted (ΔPE-Oct4-GFP) were anesthetized and the ovaries surgically exposed, including temporary removal of the ovarian bursas. The micropipette containing the wild-type donor cell suspension was placed into the exposed ovaries, and the cell suspension containing ovarian somatic cells was injected. The ovarian bursas were then replaced, and the ovaries were allowed to settle into the body cavities. The surgical sites were stapled or sutured, and the recipient mice were allowed to recover for 1 week.

Oocyte counts. One week post-intraovarian transplantation, the mice were euthanized and the ovaries were harvested and fixed in 4% paraformaldehyde. The ovaries were embedded in paraffin, serially sectioned, mounted on slides and de-waxed in xylenes, followed by hydration in a graded ethanol series. Antigen retrieval was performed by boiling the slides for 5 min in sodium citrate (pH 6.0), followed by blocking in TNK buffer (0.1 M Tris, 0.55 M NaCl, 0.1 mM KCl, 1% goat serum, 0.5% bovine serum albumin and 0.1% Triton-X in PBS), and then incubation with anti-GFP antibodies, followed by secondary antibody and chromogen for signal detection. Each section was visually examined for the presence of GFP-positive oocytes contained within follicles, and non-atretic resting (primordial), early growing (small, pre-antral), and antral follicles are quantified by counting. Comparisons in follicle numbers were made between animals receiving donor ovarian tissue, and control animals having received a mock injection.

### Results

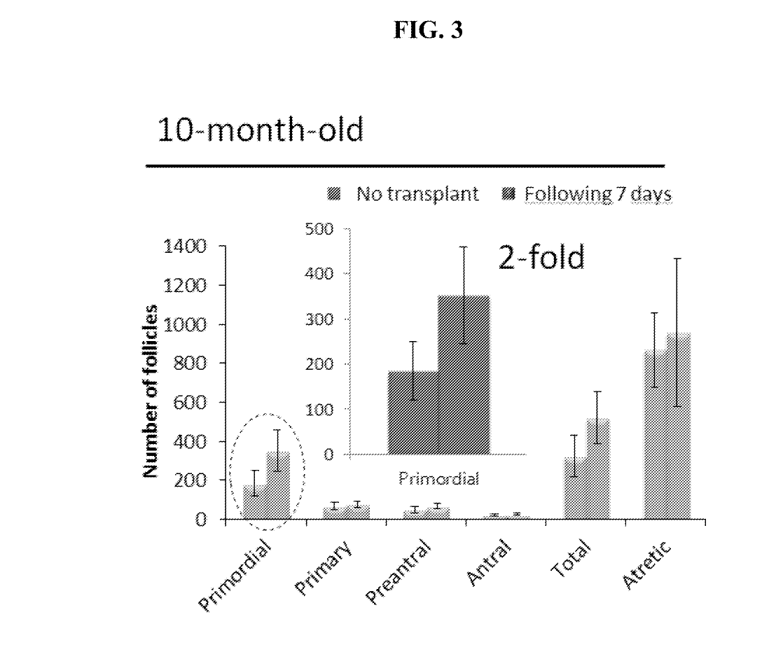

As shown in FIG. 3, reproductively aged female mice receiving intraovarian transplants of dissociated ovarian tissue-derived cells from young donors (right columns in each pair of columns), which contain an abundant number of somatic granulosa cells, the recipient primordial follicle pool increases nearly 2-fold as compared to non-transplanted controls (left columns in each pair of columns) within a week of transplant.

These results indicate that transplanted ovarian somatic cells from a source rich in follicular somatic granulosa cells work with endogenous OSCs to enable de novo follicle formation in aged ovaries. These data, combined with evidence indicating that OSCs persist in aged ovaries, while granulosa cells do not, indicate that availability of ovarian granulosa cells or their precursors represents a critical rate-limiting step to new oocyte and follicle formation by OSCs. Accordingly, the synthetic granulosa cells of the present technology are useful for rescuing or inducing follicle formation.

### Example 4

### Oogonial Stem Cells (OSCs) Persist in Peri- and Post-menopausal Human Ovaries

This example shows that OSCs are present in the ovaries of peri- and post-menopausal women and that the OSCs from post-menopausal human ovaries retain the capacity for oocyte formation ex vivo.

### Materials and Methods

Preparation of ovarian samples for flow cytometry. Ovarian cortices from de-identified female patients ranging in age from 22-58 years of age were placed into 400 U/ml collagenase type IV for use in mechanical tissue dissociator (examples include a GentleMACS or other device used for consistent mechanical dispersion) to generate a single cell suspension. The single cell suspension was washed with Hank's buffered saline solution (HBSS) followed by centrifugation (300×g) for 5 minutes. The supernatant was discarded and the cell pellet was resuspended in blocking solution consisting of HBSS supplemented with normal goat serum and bovine serum albumen. The cell suspension was incubated in the blocking solution for 30 minutes. After blocking, rabbit anti-DDX4 antibody (a germ cell linage specific antibody) was added to the cell suspension, and the cell were incubated with the antibody for 30 minutes. The cell suspension was then washed with HBSS followed by centrifugation (300×g) for 5 minutes. The cells were then incubated with a fluorescent-conjugated (such as allophycocyanin (APC) or fluorescein isothiocyanate (FITC)) goat anti-rabbit secondary antibody in preparation for flow cytometry. The cell suspension was washed HBSS followed by centrifugation (300×g) for 5 minutes to remove excess fluorescent-conjugated secondary antibody. The labeled cell suspension was loaded onto a flow cytometer, and the DDX4-positive fraction (OSCs) was determined by fluorescence. The positive events were recorded, and expressed as % yield of the total viable cell population. Woods and Tilly, Nature Protocols 8:966-988 (2013).

Culture conditions for OSCs. The DDX4-positive cell fraction obtained following flow cytometry was collected and placed into culture conditions favorable for oogonial stem cell growth, including a mouse embryonic fibroblast (MEF) feeder layer and growth medium supplemented with 10% fetal bovine serum (FBS), 1 mM sodium pyruvate, 1 mM non-essential amino acids, 1×-concentrated antibiotic solution, 0.1 mM β-mercaptoethanol, 1×-concentrated N-2 supplement, 103 units/ml LIF, 10 ng/ml epidermal growth factor, 1 ng ml⁻¹ basic fibroblast growth factor (bFGF), and 40 ng/ml glial cell-derived neurotrophic factor (GDNF). Spontaneous differentiation of human OSCs into immature oocytes was monitored by collecting culture supernatants and microscopy-based detection of oocytes. Woods and Tilly, Nature Protocols 8:966-988 (2013).

### Results

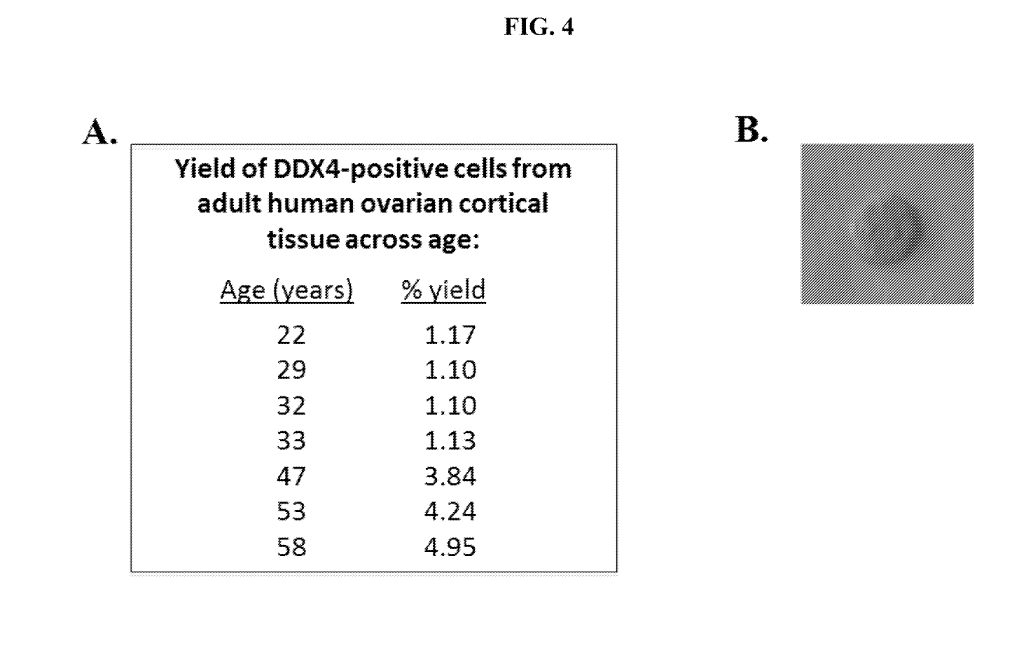

As shown in FIG. 4A-B, OSCs persist in ovaries of women at advanced ages, even after the oocyte-containing follicle pool is completely depleted in post-menopausal life (see FIG. 4A). The OSCs removed from post-menopausal human ovary tissue and cultured in vitro can still differentiate into immature oocytes (see FIG. 4B).

These results show that OSCs from aged human ovaries can still make oocytes in vitro, but the intraovarian environment in aged women is unable to support the formation of new oocytes and follicles from these cells. Accordingly, introduction of purified OSCs into human ovarian tissue that is already incapable of supporting new oocyte and follicle production will not produce new immature oocytes or follicles. These results show that the synthetic granulosa of the present technology will be useful for the support and formation of new oocytes and follicles in humans.

### Example 5

### Granulosa Cells Derived from Multi-potent Cells Produce Ovarian Steroidal Hormones

This example shows that granulosa cells differentiated from multi-potent cells produce ovarian steroidal hormones, which are needed in the formation of mature follicles and to support maturation of immature oocytes.

### Materials and Methods

To identify and track ovarian somatic cells in differentiating ESC cultures, the expression of the early granulosa cell marker, Fox12, in differentiating ESC cultures was mapped. The mapping revealed activation of the Fox12 gene by day 5. A 739 by region of the Fox12 gene promoter was identified using Genome Vista. The region was isolated from mouse genomic DNA and cloned into the pDsRed2-1 vector (Clontech, Mountain View, Calif.,) or the pLenti6 lentiviral construct containing the complete open reading frame of DsRed (Gateway Lentiviral System; Invitrogen), thus creating a DsRed expression vector under control of the Fox12 gene promoter.

Promoter activity and specificity were verified using mouse granulosa cells as a positive control and 293 cells (Invitrogen) as a negative control. To verify the Fox12 gene promoter-driven DsRed expression, undifferentiated TgOG2 ESCs were stably transfected with the Fox12-pDsRed2-1 construct via electroporation, followed by clonal selection and expansion. Alternatively, ESCs were virally transduced following initiation of differentiation using viral supernatant produced by 293 cells transfected with the Fox12-DsRed lentiviral construct (pLenti6-Fox12-DsRed). Cells were analyzed for expression of DsRed by fluorescence microscopy and isolated by fluorescence-activated cell sorting (FACS).

For FACS, differentiating ESCs were removed from the plate by either 0.25% trypsin-EDTA (prior to day 10 of differentiation) or manually scraped. The cells were then incubated with 800 U/ml of type IV collagenase (Worthington, Lakewood, N.J.) with gentle dispersion for 15 minutes followed by incubation with 0.25% trypsin-EDTA for 10 minutes to obtain single cell suspensions (after day 10 of differentiation). Cells were prepared for FACS by resuspension in 1×-concentrated phosphate-buffered saline (PBS) containing 0.1% FBS and filtration (35-μm pore size). The cells were analyzed and sorted using a FACS Aria flow cytometer (BD Biosciences, San Jose, Calif.).

Estradiol and progesterone concentrations were measured in culture medium from FACS-purified Fox12-DsRed-positive cells that had been re-plated and cultured for 24, 48 or 72 hours in the presence of PBS (vehicle), 100 ng/ml follicle stimulating hormone (FSH; NIDDK, NIH, Bethesda, Md.) or 1 mM 8-bromoadenosine-3′,5′-cyclic monophosphate (8-br-cAMP; Sigma-Aldrich). Androgen substrate necessary for aromatization to estrogen was provided by the presence of heat-inactivated 15% FBS in all cultures, which contained 0.92 pg/ml androgen (mean of 56 lots of FBS tested). The estradiol ELISA was from Alpco (Salem, N.H.), and the progesterone ELISA was from DRG International (Mountainside, N.J.). All assays were performed according to the manufacturer's guidelines.

### Results

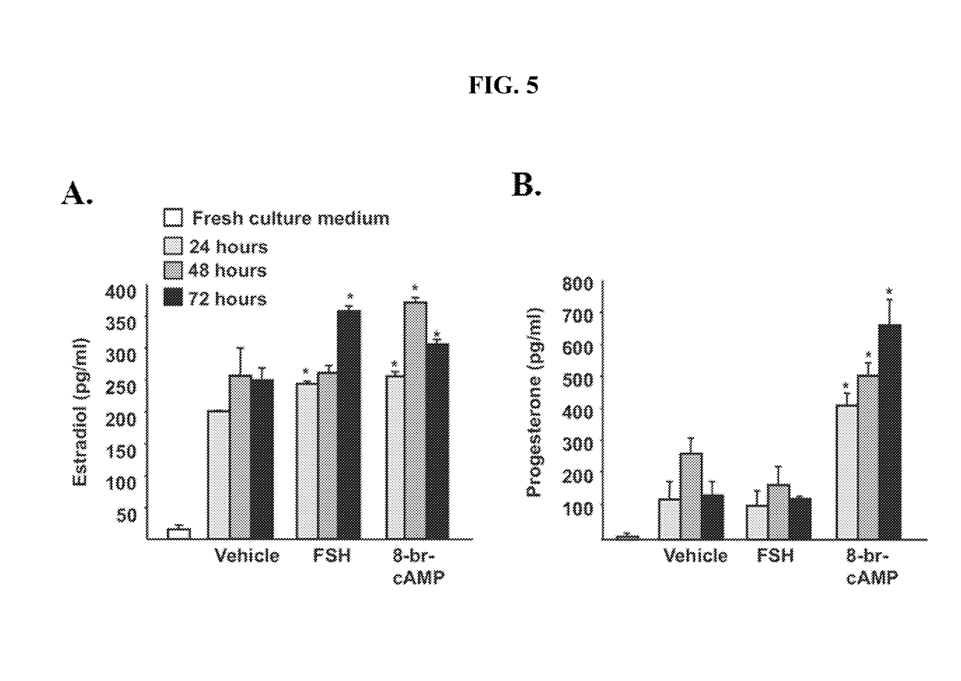

Evaluation of steroidogenesis following subculture of DsRed-positive cells isolated on day 12 of ESC differentiation revealed the presence of both estradiol and progesterone in the culture medium (FIG. 5A-5B). Additionally, the treatment with either FSH or 8-br-cAMP led to a significant increase in estradiol production, which confirmed the presence of functional FSH receptors and cAMP-mediated signaling coupled to steroidogenesis in these cells. However, only 8-br-cAMP was able to significantly enhance progesterone production (FIG. 5B).

These results show that multi-potent stem cell cultures allowed to spontaneously differentiate lead to a small number of Fox12-dsRed-expressing cells to spontaneously appear. These cells exhibit the two primary functional attributes of endogenous granulosa cells in developing ovarian follicles: FSH-responsiveness and steroidogenic capacity. These results indicate that the synthetic granulosa cells of the present technology contain functional attributes to develop ovarian follicles. Accordingly, the synthetic granulosa of the present technology are useful for the ex vivo or in vivo formation of follicles, which assist in the production of mature follicles and oocytes.

### Example 6

### Intraovarian Transplantation of Granulosa Cells

This example shows granulosa cells derived from multi-potent cells migrate to immature oocytes and developing follicles in neo-natal ovaries.

### Materials and Methods

Wild-type C57BL/6 female mice (Charles River Laboratories, Wilmington, Mass., USA) were used in the following experiments.

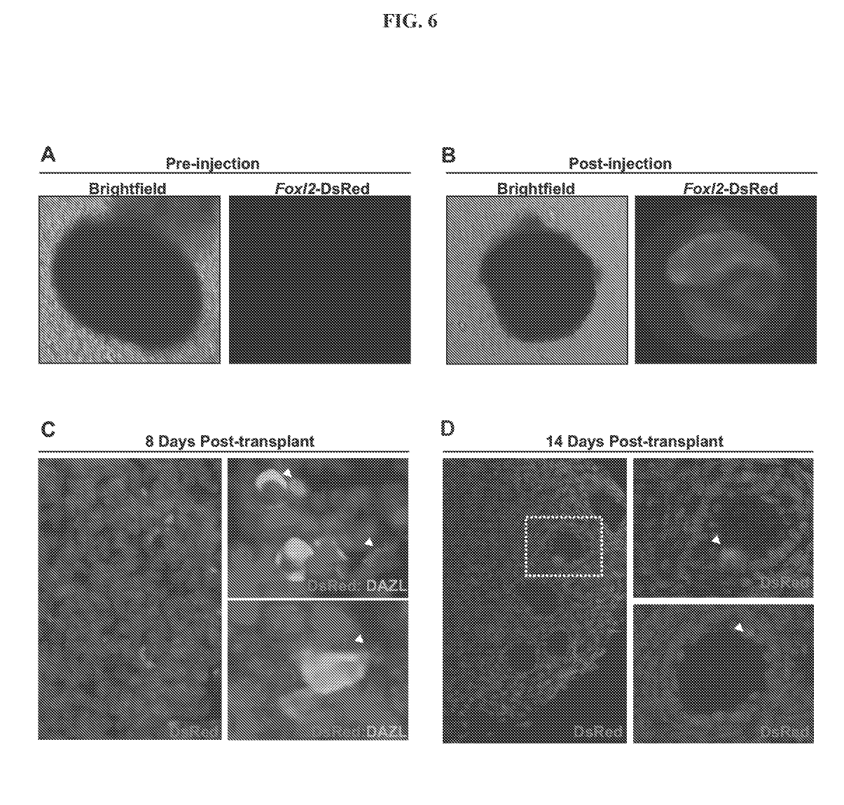

Following differentiation of Fox12-DsRed-expressing ESCs for 12 days, FACS was used to isolate DsRed-positive cells (see Example 5 for description of formation of Fox12-DsRed-expressing ESCs). For each experiment, 200-500 DsRed-positive cells were microinjected into a single neonatal (day 2-4 postpartum) wild-type mouse ovary using a Pneumatic PicoPump (World Precision Instruments, Sarasota, Fla.) (FIG. 6A-6B). Injected ovaries were then transplanted under kidney capsules of ovariectomized wild-type female mice at 6 weeks of age. At 8 days and 2 weeks post-transplantation, the grafted ovaries were removed and fixed in 4% paraformaldehyde (PFA) for analysis.

Fixed ovaries were embedded in paraffin, serially sectioned, mounted on slides and de-waxed in xylenes, followed by hydration in a graded ethanol series. Antigen retrieval was performed by boiling the slides for 5 min in sodium citrate (pH 6.0), followed by blocking in TNK buffer (0.1 M Tris, 0.55 M NaCl, 0.1 mM KCl, 1% goat serum, 0.5% bovine serum albumin and 0.1% Triton-X in PBS), incubation with the desired primary antibody (1:100 dilution) overnight at 4° C., and fluor-conjugated secondary antibody (1:250 dilution, Alexa Fluor-488 or -568; Invitrogen) at 20° C. for 1 hour. Primary antibodies used were mouse anti-Dazl antibody from Serotec (MCA2336; Raleigh, N.C.) and rabbit anti-RFP antibody for detection of DsRed from Abcam (ab62341; Cambridge, Mass.). Fluorescence image analysis was performed using a Nikon Eclipse TE2000-S inverted fluorescent microscope and SPOT imaging software (Diagnostic Instruments).

### Results

Wild-type neonatal ovary before injection of Fox12-DsRed-expressing cells isolated from ESC cultures 12 days post-differentiation show no DsRed (FIG. 6A). After injection of Fox12-DsRed-expressing cells isolated from ESC cultures 12 days post-differentiation, wild-type neonatal ovary displayed DsRed (FIG. 6B).

At 8 days post-transplantation, DsRed-expressing cells were found distributed throughout the stroma of the injected ovaries. Many of these cells were observed in close proximity to immature oocytes, as indicated by dual-immunofluorescence staining for DsRed and the oocyte marker Dazl (Deleted in azoospermia-like) (FIG. 6C). At 14 days post-transplantation, DsRed-expressing cells were no longer observed in the stroma but were detected exclusively within the granulosa layer of growing follicles (FIG. 6D).

These results show that granulosa cells and granulosa cell precursors naturally migrate to developing follicles or immature oocytes. Accordingly, synthetic granulosa of the present technology are useful for promoting the growth and maturation of follicles, follicle-like structures, and immature oocytes.

### Example 7

### Human Ovarian Cortical Strips Sustain Follicle Development Ex Vivo

This example shows that microthin ovarian cortical strips can maintain follicle formation, growth and maturation in vitro.

Materials and methods

Cortical strip culture. Young adult human ovarian tissue was dissected into microthin strips (2 mm×2 mm×1 mm) and incubated at 37° C. in serum free medium for up to 21 days to observe primordial follicle formation and subsequent activation to the first growing (primary) stage, followed by growth and maturation into multilaminar (secondary) stages.

Analysis of follicle development. Cortical strips were collected and fixed in 4% paraformaldehyde. The fixed strips were embedded in paraffin, serially sectioned, mounted on slides and de-waxed in xylenes, followed by hydration in a graded ethanol series. Antigen retrieval was performed by boiling the slides for 5 min in sodium citrate (pH 6.0), followed by blocking in TNK buffer (0.1 M Tris, 0.55 M NaCl, 0.1 mM KCl, 1% goat serum, 0.5% bovine serum albumin and 0.1% Triton-X in PBS), and then incubation with an antibody specific for oocytes (for this example, we used anti-DDX4) followed by fluorescent-conjugated (such as fluorescein isothiocyanate (FITC)) secondary antibody to allow identification of oocytes.

### Results

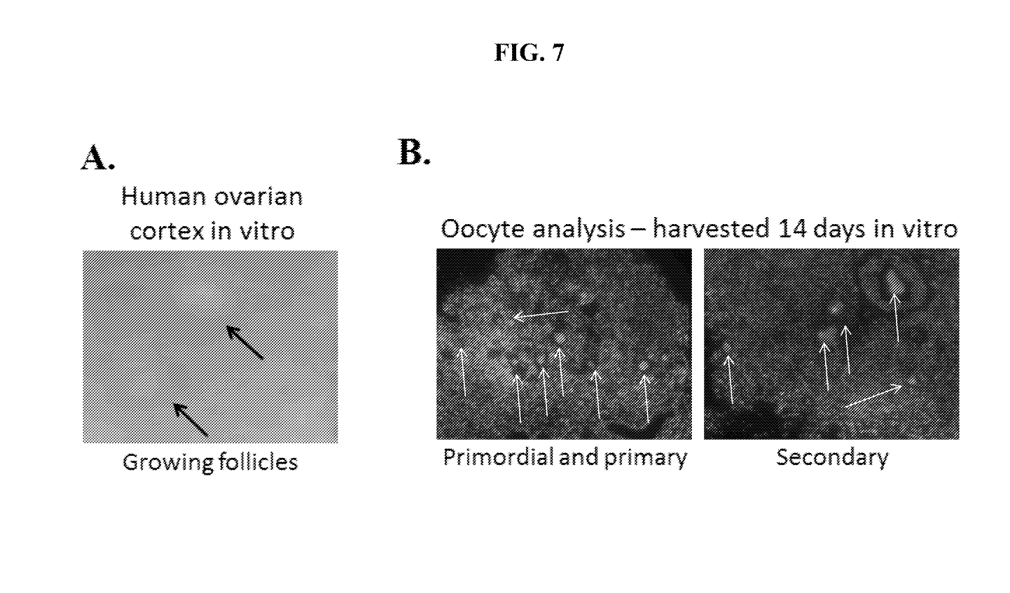

As shown in FIG. 7A, growing follicles can be visualized by light microscopy in human ovarian cortical strips cultured ex vivo for two weeks. As shown in FIG. 7B, assessment of oocytes in ovarian cortical tissue by DDX4 immunofluorescence after 14 days of ex vivo culture reveals numerous primordial and primary follicles. Right panel, detection of several multilaminar (indicated by multiple layers of granulosa cells surrounding a centrally located oocyte) or secondary follicles in cultured human ovarian cortical tissue.

The results show that immature oocyte and follicle development, as indicated by actively expanding granulosa cell layers surrounding a growing oocyte, is supported by a young adult ovarian environment ex vivo.

### Example 8

### In vitro maturation of immature oocytes to a fertilization competent stage.

This example shows that immature oocytes contained within granulosa/cumulus cell complexes harvested from preantral and early antral stage follicles contained in adult bovine ovarian cortical strips can be matured to the metaphase II (MII) stage of development ex vivo.

Materials and methods

Bovine granulosa cell/cumulus cell-oocyte complexes were collected from follicles less than 2 mm in diameter (immature, preantral stage) or greater than 3 mm in diameter (more mature, early antral stage) and placed into maturation medium at 38.5° C. for 21-24 hours to induce in vitro maturation (IVM). Maturation to the metaphase II (MII) stage (fully mature egg) was assessed by visual inspection of first polar body extrusion.

### Results

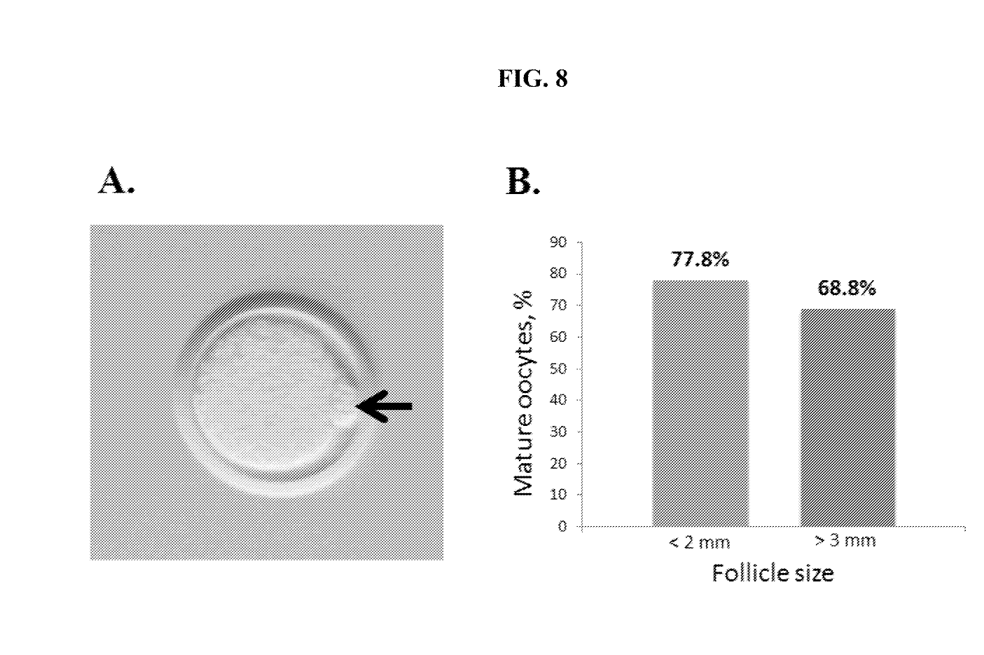

As shown in FIG. 8A, oocytes were able to mature to metaphase II, as determined by extrusion of the first polar body (polar body extrusion highlighted by arrow). Oocytes were found to mature to the MII stage of development (egg stage) at a rate of 77.8% and 68.8% from the <2 mm and >3 mm follicle diameter groups, respectively (FIG. 8B).

These results show that fully mature MII eggs can be obtained with a very high degree of success by in vitro maturation of granulosa-oocyte complexes isolated from very small preantral stage follicles present in ovarian cortical strips.

### Equivalents

The present technology is not to be limited in terms of the particular embodiments described in this application, which are intended as single illustrations of individual aspects of the present technology. Many modifications and variations of the present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

1. A method for directed differentiation of multi-potent cells into granulosa cells and/or granulosa precursor cells comprising: culturing multi-potent cells in culture conditions that direct the multi-potent cells to differentiate to granulosa cells and/or granulosa precursor cells, wherein the culture conditions comprise the absence of MEFs and LIF, without or with the presence of a GSK inhibitor.

2. The method of claim 1, wherein the culture conditions further comprise the presence of bone morphogenetic protein (BMP4) and/or retinoic acid (RA).

3. The method of claim 1, wherein the multi-potent cells contain a granulosa cell-specific reporter, wherein expression of the granulosa cell-specific reporter is indicative of a cell that is a granulosa cell or a granulosa cell precursor.

4-6. (canceled)

7. A method for directed differentiation of multi-potent cells into granulosa cells and/or granulosa precursor cells, comprising:
culturing multi-potent cells in culture conditions that direct the multi-potent cells to granulosa cells and/or granulosa precursor cells, wherein the conditions comprise the absence of MEFs and LIF, without or with the presence of a GSK inhibitor, wherein the multi-potent cells are engineered to contain one or more inducible granulosa cell-specific genes;
inducing expression of the one or more ovarian granulosa cell-specific genes; and
forming synthetic granulosa cells.

8. (canceled)

9. The method of claim 7, wherein the multi-potent cells contain a granulosa cell-specific reporter, wherein expression of the granulosa cell-specific reporter is indicative of a cell that is a granulosa cell or a granulosa cell precursor.

10. (canceled)

11. An ex vivo artificial ovarian environment comprising:
synthetic granulosa cells, wherein the synthetic granulosa cells are generated using the method of claim 1;
oocyte precursor cells; and
ovarian tissue.

12. The ex vivo artificial ovarian environment of claim 11, wherein the synthetic granulosa cells, the oocyte precursor cells, and ovarian tissue are autologous.

13. A method for making a mature follicle and a mature oocyte, comprising:
directing differentiation of multi-potent cell to granulosa cells and/or granulosa precursor cells (synthetic granulosa cells) using the method of claim 1;
combining the synthetic granulosa cells with oocyte precursor cells and ovarian tissue; and
culturing the combination of synthetic granulosa cells with oocyte precursor cells and ovarian tissue under conditions suitable to form the mature follicle and mature oocyte.

14. (canceled)

15. A method for growth and maturation of follicles and immature oocytes in ovarian tissue in a subject in need thereof, comprising contacting ovarian tissue with synthetic granulosa cells, wherein the synthetic granulosa cells are made using the method of claim 1.

16-18. (canceled)

19. A method for increasing levels of one or more ovarian derived hormones and growth factors in a subject in need thereof, the method comprising:
directing differentiation of multi-potent cells to granulosa cells and/or granulosa precursor cells (synthetic granulosa cells) using the method of claim 1;
isolating an enriched population of synthetic granulosa cells based on expression of a granulosa cell-specific reporter; and
administering an effective amount of the enriched population of synthetic granulosa cells to the subject, wherein the granulosa cells or granulosa cell precursors secrete one or more ovarian derived hormones and growth factors, and wherein after administration of the enriched population of synthetic granulosa cells the subject displays elevated levels of one or more ovarian derived hormones and growth factors as compared to before administration of the enriched population of synthetic granulosa cells.

20. The method of claim 19, further comprising stimulating the synthetic granulosa cells to secrete ovarian derived hormones and growth factors.

21. (canceled)

22. (canceled)

23. The method of claim 19, wherein the synthetic granulosa cells are autologous to the subject.

24. The method of claim 23, wherein the subject is human.

25. An ex vivo method for producing mature follicles and mature oocytes, comprising:
combining synthetic granulosa cells, oocyte precursor cells, and ovarian tissue; and
culturing the combination of synthetic granulosa cells, oocyte precursor cells, and ovarian tissue under conditions sufficient to produce mature follicles and a mature oocyte, wherein the synthetic granulosa cells are made using the method of claim 1, and the synthetic granulosa cells, the oocyte precursor cells, and the ovarian tissue are autologous.

26. The method of claim 25, wherein the oocyte precursor cells are derived from multi-potent cells, female germ line stem cells, or oogonial stem cells.

27. (canceled)

28. The method of claim 26, wherein the multi-potent cells, female germ line stem cells, or oogonial stem cells are genetically modified to correct a gene defect or to express a desired gene.

29. (canceled)

30. A method for developing genetically modified mature oocytes for a subject diagnosed with a genetic disease, comprising:
genetically modifying multi-potent cells, female germ line stem cells, or oogonial stem cells from the subject to correct a gene defect;
culturing the multi-potent cells, female germ line stem cells, or oogonial stem cells under conditions sufficient to produce oocyte precursor cells;
combining the oocyte precursor cells with synthetic granulosa cells and ovarian tissue, wherein the synthetic granulosa cells are made using the method of claim 1, and the synthetic granulosa cells and ovarian tissue are autologous to the subject; and
culturing the combination of synthetic granulosa cells, oocyte precursor cells, and ovarian tissue under conditions sufficient to produce mature follicles and a mature oocyte, wherein the mature oocyte does not carry the genetic disease.

31. (canceled)

32. A method for producing mature oocytes ex vivo, comprising:
combining synthetic granulosa cells, oocyte precursor cells, and ovarian tissue; and
culturing the combination of synthetic granulosa cells, oocyte precursor cells, and ovarian tissue under conditions sufficient to produce mature follicles and a mature oocyte, wherein the synthetic granulosa cells are made using the method of claim 1, and the synthetic granulosa cells, the oocyte precursor cells, and the ovarian tissue are autologous.

33. The method of claim 32, wherein the oocyte precursor cells are derived from multi-potent cells, female germ line stem cells, or oogonial stem cells; or the oocyte precursor cells are primordial germ cells, female germ line stem cells, or oogonial stem cells.

34. (canceled)

35. The method of claim 33, wherein the multi-potent cells, female germ line stem cells, or oogonial stem cells are genetically modified to correct a gene defect or express a desired gene.

36. (canceled)

37. (canceled)
